(19) United States
(12) Patent Application Publication
(10) Pub. No.: US 2007/0270690 A1
(43) Pub. Date: Nov. 22, 2007

(54) NON-CONTACT MEDICAL REGISTRATION WITH DISTANCE MEASURING

(76) Inventor: Swen Woerlein, Munich (DE)

Correspondence Address: DON W. BULSON (BrainLAB), RENNER, OTTO, BOISSELLE & SKLAR, LLP, 1621 EUCLID AVENUE - 19TH FLOOR, CLEVELAND, OH, 44115, US

(21) Appl. No.: 11/750,353

(22) Filed: May 18, 2007

Related U.S. Application Data
(60) Provisional application No. 60/803,304, filed on May 26, 2006.

(30) Foreign Application Priority Data
May 18, 2006 (EP) 06010292

(51) Int. Cl. A61B 5/05

(52) U.S. Cl. 600/429

(57) ABSTRACT

A medical registration device includes a localizing device for detecting a spatial position of a treatment device, treatment-assisting device, patient or patient body part, said localizing device including a data processing unit operative to assign detected spatial positions of the patient or patient body part to corresponding points of an acquired patient image data set, and a distance measuring device communicatively coupled to the data processing unit. A spatial position of the distance measuring device is detected by the localizing device, and the distance measuring device transfers distance data for measured points to the data processing unit. Based on the detected spatial position and the measured distance, a spatial location of the point can be ascertained.

### RELATED APPLICATION DATA

This application claims priority of U.S. Provisional Application No. 60/803,304 filed on May 26, 2006, which is incorporated herein by reference in its entirety.

### FIELD OF THE INVENTION

The invention relates to non-contact medical registration and, more particularly, to a medical registration device and method for non-contact medical registration of patients and/or patient parts.

### BACKGROUND OF THE INVENTION

In order to provide visual assistance to physicians during treatment, image data are often used that are ascertained using so-called medical imaging methods. Examples of such imaging methods are computer tomography, nuclear spin tomography, x-ray methods, PET and SPECT. Using these methods, an image data set of the patient can be obtained. In most cases the image data set is a three-dimensional image data set with internal and external structures mapped therein. In order to use this data set during treatment (e.g., in order to display treatment devices or treatment-assisting devices in a correct positional relationship to said image data set), a so-called registration is performed in situ (i.e., during or just before treatment). In such a registration, the structures from the image data set and corresponding points on or in the patient are assigned to each other and defined in a specific spatial coordinate system. Instruments or other treatment means then can be visually displayed in a correct positional assignment to acquired image data, and as a result the physician performing the treatment can be provided with image assistance.

In order to perform such registration, it is in principle possible to move a pointing instrument, which is already registered, to particular points (also called landmarks) on a patient, e.g., using the tip of a pointer, and to then communicate to an assisting computer system, which in most cases is a medical navigation system, which point in the image data set corresponds to the currently identified point. Once multiple points have been identified and assigned, a three-dimensional registration can be performed. Such conventional registration method, however, can be complex and time-consuming. Further, the method also requires optical or magnetic tracking (positional tracking) in the navigation system.

A registration method is known from U.S. Pat. No. 6,033,415 that is intended to serve in performing robotic orthopaedic procedures, wherein a pointer tool (e.g., a digitization means) arranged on a robotic jointed arm is used to identify a number of points on a bone, and a robot, which is equipped with joint sensors, detects the external shape of the bone in its coordinate system. This external shape then can be used to transform a previously acquired mapping of the bone onto the detected actual position. Producing digitized bone data sets via a non-contact digitization apparatus (e.g., ultrasound or laser system) attached to a robot is also proposed. Details or practical implementations for applications, however, are not specified.

A major disadvantage of contact registration using the tip of a robotic arm, as described above is that it incurs a relatively high degree of complexity in identifying all of the necessary points. The embodiment shown for non-contact registration or digitization is problematic alone for the reason that the digitization apparatus, which is arranged fixedly on the robot, can only see part of the patient, and can only see this part from a single perspective. Such non-contact registration method also is not practicable, since for the aforesaid reason, many different body parts cannot be detected.

Another non-contact registration method is known from EP 1 142 536 B1, wherein a patient is registered in a medical navigation system by means of light points radiated onto the patient. Because the light points on the surface of the patient serve as registration points themselves, they must be as clearly visible to the tracking system, which proves difficult in some applications.

### SUMMARY OF THE INVENTION

A medical registration device includes a localizing device, by means of which the spatial position of treatment devices, treatment-assisting devices, patients or patient parts can be detected. The localizing device includes all means that can establish a spatial location of the patient, treatment devices or treatment-assisting devices (e.g., a position in three-dimensional space of an instrument or its tip). The registration device can further include a data processing unit that assigns detected positions of patients or patient parts to corresponding points of an acquired patient image set. The registration device can include a distance measuring device, the spatial position of which can be detected by the localizing device. The distance measuring device can transfer the distance data for measured points to the data processing unit.

When registering an object, it is the distance measuring that allows points to be detected without having to move an instrument directly to the points. Such distance measuring devices are already commercially available or can be readily adapted to the medical registration device. Because the spatial position of the distance measuring device can be detected by the localizing device, the distance measuring device can be provided in a non-fixed embodiment (i.e., the distance measuring device can be moved or handled). This enables points or areas that are not visible to an optical tracking system (e.g., points or areas within a resection region or other optically “undercut” areas) to be registered.

The localizing device can be a medical tracking system, in particular an optical tracking system and specifically a camera tracking system (e.g., a stereoscopic camera unit), in which positions of treatment devices, treatment assisting devices, patients, or patient parts may be determined via reference arrays attached thereto. It is noted that the tracking system need not record the points for registration, but may merely detect the distance measuring device, wherein problems with visibility of the distance measuring device rarely arise. A reference array, for example, can be arranged on the distance measuring device for this purpose.

The distance measuring device also can be arranged on a jointed arm, wherein the localizing device includes position determining sensors or change-in-position sensors, in particular angle detecting sensors, which can be arranged in the joints of the jointed arm. The jointed arm can be a robotic arm of a medical robot. Such localization with the aid of joint sensors can be used on its own, or a localizing device can be used that includes both an arm with joint sensors and optical tracking of the distance measuring device. The latter embodiment enables mutual redundant supplementing of the measured distance.

The data processing unit can be a part of a medical navigation system, and such medical navigation systems are generally present during surgical procedures.

The distance measuring device, for example, can be a laser beam distance meter, in particular a laser beam distance meter for measuring a linear distance. Such devices are available, simple in design and can be easily integrated into the registration system as describe herein. In principle, the distance measuring device simply provides data on the distance of a point, and many devices can also provide such function. Therefore, any distance determining system can be used, including a focal distance system of a spatially localizable medical microscope. An auto-focus means of such a microscope, which focuses on a particular point, for example, can be adduced for this purpose. This auto-focus point or auto-focus distance is known in the microscope system and can be used as a registration distance. If a microscope is used that includes sensors that enable its position or the position of its functional parts to be determined in the spatial coordinate system, the distance can be measured by means of focussing, even without an external tracking system.

In accordance with another aspect of the invention, there is provided a method for the medical registration of patients and/or patient parts with respect to corresponding points of an acquired patient image data set. The spatial position of a distance measuring device can be detected by a localizing device, and the distance data for measured points can be transferred to a data processing unit that ascertains the spatial position of the points from the spatial position of the distance measuring device and the distance data. The advantages which can be achieved by the method correspond to those discussed above with respect to the corresponding registration device.

Registration can be performed by a separate data processing unit or by the data processing unit already described above, wherein with the aid of a surface matching method, points can be assigned between the acquired image data set and multiple points, the spatial position of which have been ascertained. Such surface matching methods or programs are known and available and can exactly assign the structures from the distance detection and from the acquired image data set on the basis of specifically shaped surfaces. Such registration is of course generally possible for previously acquired image data sets, but also for image data sets acquired during the treatment.

The localizing device can be a medical tracking system such as has already been described above, and similarly the spatial position of the distance measuring device can be detected by a reference means arranged thereon. In terms of the method, this also results in the possibility of detecting a point as a registration starting point using the distance measuring device, said point being assigned to a reference array that is attached to the patient or is in a known positional relationship to the patient. This makes it easier to initially assign points for registration. It should be noted that a so-called point-to-point registration also can be performed using the distance measuring device (e.g., by detecting the distance for particular landmarks and registering as described herein).

The spatial position of the points can be detected by means of a distance measuring device that is arranged on a jointed robotic arm of a medical robot that includes position determining sensors or change-in-position sensors, in particular angle detecting sensors, in the joints of the jointed arm. There further exists the possibility of automatically or semi-automatically detecting the position by controlling the jointed arm of the robot, wherein the position of a sufficient number of points within a predefined target region may be consecutively ascertained, by means of which registration can be performed.

The method and device described herein allows a patient to be registered to previously or intra-operatively acquired image data sets, by using a calibrated non-contact distance measuring device, for example a so-called laser range finder. The distance measuring device can measure the distance from a point on the surface of the patient's body, while the relative position of the distance measuring device in relation to the patient is known or may be determined (for example by a tracking system which tracks the position of the distance measuring device and of the patient). The information on the points already acquired (for example spreading, differentiation, etc.) can be used to establish whether a sufficient number of points for a successful registration have already been acquired. By repeating this process, multiple points having known positions relative to the patient can be detected, and these points then can be used as a part of the input of a surface matching algorithm, the other part of the input being the patient data set. The algorithm then can perform matching (e.g., adapt the points to the data of the data set), and the positional relationship of the patient to the image data set is obtained.

In order to provide automatic or semi-automatic registration, the distance measuring device can be mounted on the robotic arm. The robotic arm then can be positioned in such a way that the distance measuring device points to a region of interest on the surface to be registered. This region of interest also can be determined automatically, for example by using a localization device (reference array) that is fastened to the patient and used as a starting position. The robotic arm then can be moved in relation to the predefined position, for example in a radius around the predefined position, and the distance measuring device can be triggered to detect registration points. The process can be repeated for multiple regions of interest. The information on the registration points already acquired can in turn be used to determine whether a sufficient number of points for a successful registration have been acquired, and the acquisition process also can be automatically concluded in order to start a surface matching method.

When the robotic arm has its own coordinate system, wherein the spatial relationship of different positions is known, and the patient is in a fixed (but not necessarily known) position with respect to the robotic coordinate system, “external tracking” can be omitted. “Internal tracking”, provided by the known joint positions of the robot, is sufficient, and by using the information of the distance measuring device, the systems can determine the positions of the acquired points in the robotic coordinate system. If the positional relationship of the patient to the image data set has then been determined by surface matching, the positional relationship to the robotic coordinate system is likewise known, and it is possible to navigate.

### BRIEF DESCRIPTION OF THE DRAWINGS

The forgoing and other features of the invention are hereinafter discussed with reference to the drawings.

FIG. 1 is a schematic view of an exemplary registration system in accordance with the invention, wherein the distance measuring device can be freely guided.

FIG. 2 is a schematic diagram of an exemplary registration system in accordance with the invention, wherein an externally tracked distance measuring device is located on a robotic arm.

FIG. 3 is a schematic diagram of an exemplary registration system in accordance with the invention, without an external tracking system.

### DETAILED DESCRIPTION

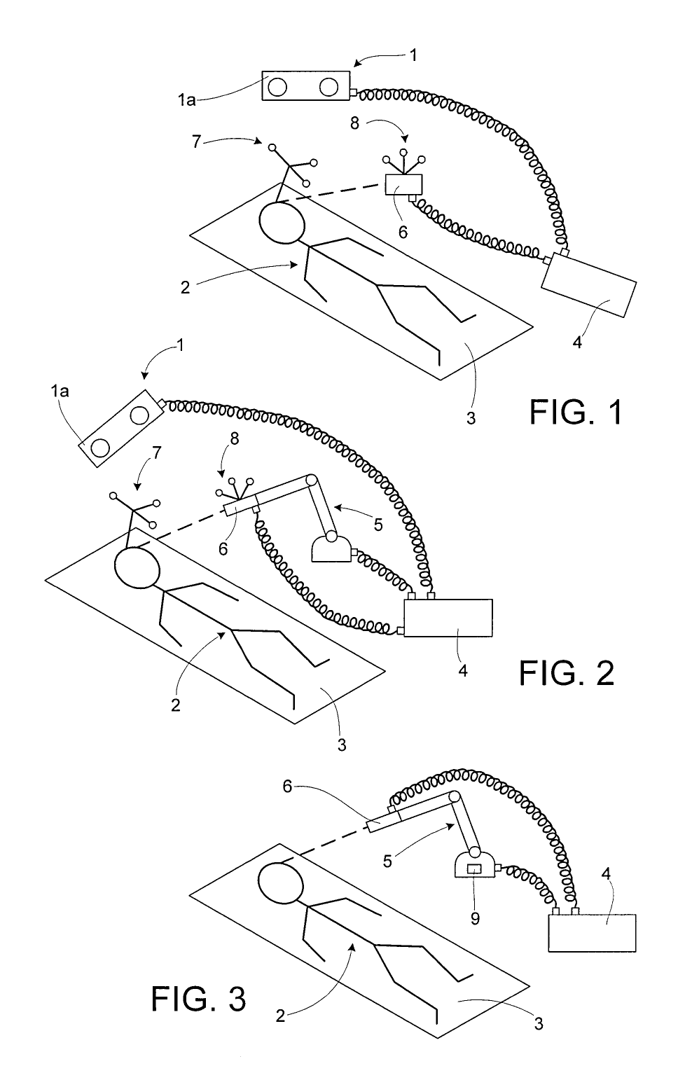

The schematic representation in FIG. 1 shows an exemplary registration system, wherein a patient 2 is lying on a table 3, and a reference array 7 that forms part of an optical tracking system 1 is arranged on the patient 2 in a region to be registered. The optical tracking system 1 (e.g., an “external” tracking system) also includes a stereoscopic camera unit 1a and logic that can assign spatial positions to observed points such as, for example, reference spheres on the reference array 7. A distance measuring device 6 also is shown which, in the present example, may be a laser distance meter. The broken line indicates that the distance between the distance meter 6 and a point on the patient's head is measured. The laser distance meter 6 also may be provided with a reference star (reference array) 8, and thus a spatial position of the distance meter 6 can be established by the tracking system 1. Both the tracking system 1 and the laser distance meter 6 are connected to a data processing unit 4, which is shown separately but can be the data processing unit of a medical navigation system (not shown).

The distance meter 6 can be arranged such that it can be freely moved, e.g., the distance meter 6 can be guided by hand, in order to register a particular point. This allows points on the patient 2 that are poorly visible, for example within an incision that already has been made, to also be registered. Because the position of the distance meter 6 is known in the system via the reference array 8, and the linear distance from each point (broken line) can be measured and relayed to the data processing unit 4, it is also possible to establish the current position in the spatial coordinate system of the point whose distance is currently being measured. If a sufficient number of points have been detected in this manner, the structure thus mapped can be assigned to a corresponding structure of an image data set previously acquired by means of an imaging method. This enables non-contact registration, which can be configured to be simple, even for poorly accessible points.

FIG. 2 shows another embodiment of a registration device, wherein identical reference signs in the figures indicate identical devices. In this system, the distance meter 6 is fastened to the end of the arm of a jointed arm robot 5. Using the robot 5 allows patient registration points to be automatically or semi-automatically acquired. It is then possible for the robotic arm, starting from a point determined using reference array 7, to automatically record a number of points in a region of interest, or once activated with the aid of the distance meter 6, by automatically moving to multiple points in said region, wherein the aforesaid point determined using the reference array 7 defines a predetermined starting point. If enough points on the patient have been acquired to allow them to be assigned to corresponding image data in the image data set, the robot can automatically stop acquiring patient registration points or can continue the same process at another, also predefined region of interest.

Another aspect of using the robot relates to its own internal coordinate system. There are medical robots available that have a spatial coordinate system and which, starting from a zero point, can track the movements of their arm sections or the functional means attached thereto (e.g., via joint sensors in the joints between the arms or between the first arm portion and the robotic base). Such a robot thus knows where, in its own coordinate system (the “internal” tracking system) where the distance measuring device 6 lies and, via the distance measured there, also where the patient point currently being acquired lies. If “external tracking” is also performed via the tracking system 1 and the reference array 8 on the distance meter 6, these two localizing systems can redundantly supplement each other, or if one system fails, it is possible to fall back on the data of the other system. For this purpose, the coordinate systems of the robot 5 and the tracking system 1 can be matched.

Since robots can include a localizing device of their own having joint sensors, embodiments are also conceivable such as are shown in FIG. 3, for example. This embodiment does not use an external tracking system, but simply just the joint coordinate system of the robot. By means of a data processing unit 9, positional data for the distance meter 6 can be processed and, with the aid of the distance relayed from the distance meter 6, positional data on the currently calibrated point on the patient also can be processed. This data can be provided to the data processing unit 4.

It is likewise possible, as also shown in FIG. 3, for the data processing unit 4 and the distance meter 6 to be directly connected. In this case as well, the data processing unit 4 receives all the data necessary for registering points that have been moved to or calibrated onto corresponding structures in an image data set.

In such a method, the position of the patient need not be known in the system. If the patient is properly fixed, patient points can be acquired and registration performed in the way described above, and the distance measuring device 6 on the robot 5 then could be replaced by a medical instrument with which it is possible to work in a navigated way with image assistance.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A medical registration device, comprising:
a localizing device for detecting a spatial position of a treatment device, treatment-assisting device, patient or patient body part, said localizing device including a data processing unit operative to assign detected spatial positions of the patient or patient body part to corresponding points of an acquired patient image data set; and
a distance measuring device communicatively coupled to the data processing unit, wherein a spatial position of the distance measuring device is detectable by the localizing device, and the distance measuring device is operative to transfer distance data for measured points to the data processing unit.

2. The registration device according to claim 1, wherein the data processing unit is operative to ascertain a spatial position of at least one point of interest based on a measured distance from the distance measuring device to the point of interest and an ascertained spatial position of the distance measuring device.

3. The registration device according to claim 1, wherein the localizing device is a medical tracking system operative to track a spatial position of a reference array attached to the treatment device, treatment assisting device, patient or patient body part.

4. The registration device according to claim 3, wherein the medical tracking system is a camera tracking system,

5. The registration device according to claim 3, wherein the reference array is arranged on the distance measuring device.

6. The registration device according to claim 1, further comprising a mechanical arm including at least one joint, and the distance measuring device is arranged on the mechanical arm, wherein the localizing device includes at least one position sensor operative to detect position data for the at least one joint.

7. The registration device according to claim 6, wherein the position sensor is an angle detecting sensor.

8. The registration device according to claim 6, wherein the mechanical arm is a robotic arm of a medical robot.

9. The registration device according to claim 1, wherein the data processing unit is a part of a medical navigation system.

10. The registration device according to claim 1, wherein the distance measuring device is a laser beam distance meter for measuring a linear distance.

11. The registration device according to claim 1, wherein the distance measuring device is a focal distance determining system of a spatially localizable medical microscope.

12. A method for the medical registration of patients and/or patient parts with respect to corresponding points of an acquired patient image data set, comprising:
using a distance measuring device to detect a distance to at least one point of interest;
detecting a spatial position of the distance measuring device;
ascertaining a spatial position of the at least one point of interest based on the measured distance to the point and the ascertained spatial position of the distance measuring device.

13. The method according to claim 12, wherein detecting the spatial position of the distance measuring device includes using a localizing device to detect the spatial position.

14. The method according to claim 12, wherein the at least one point of interest is a plurality of points of interest, further comprising registering the plurality of points of interest to corresponding points in the patient image data set.

15. The method according to claim 14, wherein registering includes using a surface matching method to assign the plurality of points of interest to corresponding points of the patient image data set.

16. The method according to claim 12, wherein detecting the spatial position of the distance measuring device includes using a medical tracking system operative to determine a spatial position of a reference array attached to a treatment device, a treatment-assisting device, the patient or patient parts.

17. The method according to claim 16, wherein using a medical tracking system includes using a camera tracking system that includes a stereoscopic tracking camera unit.

18. The method according to claim 16, wherein detecting the spatial position of the distance measuring device includes using a reference array attached to the distance measuring device.

19. The method according to claim 16, further comprising using the distance measuring device to detect a point as a registration starting point, said point being assigned to a reference array that is attached to the patient or is in a known positional relationship to the patient.

20. The method according to claim 12, further comprising arranging the distance measuring device on a jointed robotic arm, said arm including at least one position sensor operatively coupled to a joint of the jointed robotic arm, and wherein the spatial position of the distance measuring device is based on data obtained from the at least one position sensor.

21. The method according to claim 20, wherein ascertaining the spatial position includes automatically or semi-automatically commanding the jointed robotic arm to ascertain a predetermined number of points within a predefined target region.
